(19) United States
(12) Patent Application Publication
(10) Pub. No.: US 2025/0032567 A1
(43) Pub. Date: Jan. 30, 2025

(54) PLANT-BASED SKIN PREPARATIONS AND USE THEREOF

(71) Applicant: ReaGenics Research Ltd., Ness Ziona (IL)

(72) Inventor: Michael Kagan, Jerusalem (IL)

(21) Appl. No.: 18/837,861

(22) PCT Filed: Feb. 12, 2023
(86) PCT No.: PCT/IL2023/050143

§ 371 Date: Aug. 12, 2024

Related U.S. Application Data
(60) Provisional application No. 63/309,564, filed on Feb. 13, 2022.

(51) Int. Cl. A61K 36/185; A61K 9/06; A61K 9/127; A61P 17/00; C12N 5/04

(52) U.S. Cl. CPC A61K 36/3482; A61K 9/06; A61K 9/127; A61P 17/00; C12N 5/04

(57) ABSTRACT

The present invention relates to skin care preparations comprising callus-derived pluripotent cells of a plant of the genus Cannabis, including Cannabis sativa I., and Cannabis indica, compositions comprising same and use thereof for promoting skin health and appearance.

### FIELD OF THE INVENTION

The present invention relates to skin care preparations comprising callus-derived pluripotent cells of a plant of the genus Cannabis, including Cannabis sativa L. and Cannabis indica, compositions comprising same and use thereof for promoting skin health and appearance.

### BACKGROUND OF THE INVENTION

Skin is the primary interface between the body and the environment. As the skin is the largest (representing one-sixth of the total body weight) and first-contact organ of the human body, its balance and proper functionality are crucial for human health.

The skin participates in sensitivity and offers protection against microorganisms, chemicals and ultraviolet radiation. In addition, it is constantly exposed to environmental factors of UV light, pathogens, chemical threats and temperature changes. All of these coupled with the genetic background can cause severe diseases such as skin inflammation, which may develop to chronic inflammatory diseases.

Skin diseases associated with chronic inflammation are becoming more and more common nowadays, especially due to air pollution, the high hygiene level and the increasing amount of chemicals in consumed products. The use of plants in treatment of inflammatory skin diseases results from their influence on different stages of inflammation. Dawid-Pad, 2013 is a review paper presenting results of a study regarding the anti-inflammatory activity of plant raw material related to its influence on skin (Dawid-Pać, R. 2013. Postepy Dermatol Alergol. 2013 June; 30(3): 170-177). Styrezewska, et al., describe the use of natural components derived from oil seed plants for treatment of inflanmatory skin diseases. (Styrczewska M et al., 2019. Current Pharmaceutical Design, 25(20):2241-2263).

The main skin disorders associated with an inflammatory imbalance are allergic contact dermatitis, atopic dermatitis and psoriasis, which besides the loss in skin functionality have a high social and psychological impact.

Plant extracts have been widely used in pharmacy and cosmetics for the preparation of extracts comprising active ingredients for various uses. Extraction processes with particular solvents or mixtures have been standardized for various plant species.

For Example, U.S. Pat. No. 10,954,488 discloses a preparation obtained from an in vitro culture of undifferentiated cells of Mimosa pudica, as well as to the preparation method thereof; a cosmetic or dermatological composition comprising said preparation; and the uses thereof for the treatment of inflammatory skin conditions, as an antioxidant agent in the treatment of oxidative stress caused by environmental pollution, and as an anti-aging agent.

U.S. Application Publication No. 2022/0088100 discloses cosmetic, pharmaceutical, or nutraceutical use of an extract derived from cell cultures of Cannabis sativa, preferably a hydro-alcoholic extract, and a cosmetic, pharmaceutical, or nutraceutical composition comprising this extract; in particular, the composition containing this extract of Cannabis sativa may be used to reduce neurogenic inflammation and stimulate the production of neurotransmitters or neuropeptides, such as dopamine and endorphin.

However, extraction processes are typically expensive, and allow to obtain only particular fraction(s) of the plant compounds that have physicochemical characteristics suitable for use in pharmaceutical and/or cosmetic formulations.

Alternatively, plant cells cultures are used to obtain active ingredients secreted to the culture medium, either naturally or using molecular genetic techniques to manipulate the secretion of endogenous as well as heterologous compounds.

For example, U.S. Patent Application Publication No. 2018/0133138 discloses cell-free supernatants (conditioned media) that previously supported the growth of a dedifferentiated plant cell suspension culture, or a fraction of said cell-free supernatant, said cell-free supernatant or said fraction comprising peptides from 4 to 300 amino acids length and including peptide plant growth factors and peptide plant transcription factors, and without having cytoplasmic cell contents from the cell lysis and membranes and/or cell walls. Fractions of said supernatants and cosmetic applications for promoting re-youth of skin are also disclosed.

There is a constant need for additional and alternative plant-derived compositions effective in promoting skin health and/or appearance which can be obtained from plant is a reproducible manner under commercial scale.

### SUMMARY OF THE INVENTION

The present invention relates to the field of plant-based material useful in treating skin diseases and or in maintaining and/or improving overall skin appearance.

The present invention discloses compositions comprising callus derived pluripotent cells from the Cannabis plant, which maintain their capability to produce cannabinoids, and have beneficial effects on mammal skin stem cells, including protecting dermis stem cells against intrinsic and extrinsic stress factors, promoting proliferation of dermis stem cells, and protecting dermis cells from apoptosis. The compositions of the present invention are further effective in treating skin disorders, including skin inflammatory diseases.

The present invention is based in part on the use of callus-derived, pluripotent plant cells in skin care preparations, formulated preferably in nanoparticles such as liposomes; wherein these plant cells are grown in-vitro from the genus Cannabis, particularly Cannabis sativa L. and Cannabis indica, and wherein these undifferentiated, pluripotent plant cells contain all or part of the range of cannabinoids, optionally also terpenes and flavonoids, found in the naturally grown plant. Without wishing to be bound by any specific theory or mechanism of action, the unique combination of pluripotent stem cells and cannabinoids act synergistically for maintaining and promoting the appearance of healthy skin, as well as of for treating skin disorders, including skin inflammatory diseases.

According to certain aspects, the present invention provides a plant-based skin care preparation for a mammalian subject, comprising callus-derived cells of a plant of the genus Cannabis, and at least one dermatologically acceptable carrier, wherein the callus-derived cells are pluripotent cells.

According to certain embodiments, the Cannabis is selected from the group consisting of Cannabis sativa L. and Cannabis indica. Each possibility represents a separate embodiment of the present invention.

It is to be explicitly understood that the present invention encompasses various Cannabis plant varieties, cultivars, and derivatives thereof as is known in the art.

According to certain embodiments, the callus derived pluripotent cells are capable of maintaining and/or promoting at least one of the growth, vitalization, regeneration, survival, and health of mammalian skin stem cells.

According to certain embodiments, the skin stem cells are epidermal stem cells.

According to certain embodiments, the skin stem cells are dermal stem cells.

According to certain embodiment, the concentration of the callus-derived pluripotent cell is from about 0.001% to about 25% w/w based on a dry weight of the total weight of the preparation.

According to certain embodiments, the callus-derived pluripotent cells are cultured in medium, forming a cell suspension.

According to certain embodiments, the medium comprises at least one agent supporting the propagation of the callus-derived pluripotent cells.

According to certain embodiments, the medium comprises at least one agent inducing or enhancing the production of at least one secondary metabolite within the callus-derived pluripotent cells.

According to certain embodiments, the callus-derived pluripotent cells cultured in a medium are maintained under conditions inducing or enhancing the production of at least one secondary metabolite within the callus-derived pluripotent cells.

The agent or condition inducing or enhancing the production of the at least one secondary metabolite include one or more biotic, abiotic, and environmental stressors.

According to certain embodiments, the callus-derived pluripotent cells are separated from the medium.

According to certain embodiments, the callus-derived pluripotent cells comprise at least one secondary metabolite, wherein the at least one secondary metabolite is endogenous to the Cannabis plant from which said cells are obtained. The said cells can comprise all or part of the corresponding Cannabis plant secondary metabolites.

According to certain embodiments, the callus-derived pluripotent cells comprise at least one secondary metabolite selected from the group consisting of cannabinoids, terpenes, flavonoids, and any combination thereof. Each possibility represents a separate embodiment of the present invention.

According to certain preferred embodiments, the callus-derived pluripotent cells comprise at least one cannabinoid.

According to certain embodiments, the preparation comprises at least about 0.05% total cannabinoids out of the total dry weight of the callus-derived pluripotent cells.

According to certain embodiments, the preparation comprises between about 0.1% to about 3% total cannabinoids out of the total dry weight of the callus-derived pluripotent cells.

According to certain embodiments, the at least one cannabinoid is selected from the group consisting of cannabidiol (CBD), CBN (cannabinol), cannabigerol (CBG), cannabidiolic acid (CIDA), CBC (cannabichromene), CBL (cannabicyclol), CBV (cannabivarin), cannabidivarin (CBDV), cannabigervarin (CBGV), CBCV (cannabichromevarin), CBGV (cannabigerovarin), CBGM (cannabigerol monomethyl ether), CBE (cannabielsoin), CBT (cannabicitran), tetrahydrocannabinol (THC) tetrahydrocannabivarin (THCV), and any combination thereof. Each possibility represents a separate embodiment of the present invention.

According to certain embodiments, the profile of the cannabinoids within the callus-derived pluripotent cells of the invention resembles the profile of said cannabinoids in the Cannabis tissue from which the callus is formed.

According to certain embodiments, the callus-derived pluripotent cells comprising at least one cannabinoid further comprises at least one terpene. According to certain embodiments, the callus-derived pluripotent cells comprising at least one cannabinoid further comprises at least one flavonoid.

According to certain embodiments, the callus-derived pluripotent cells of the invention comprise a combination of at least one cannabinoid, at least one terpene and at least one flavonoid. According to these embodiments, the least one cannabinoid, at least one terpene and at least one flavonoid act synergistically in at least one of maintaining skin appearance, promoting skin appearance, treating a skin disorder or a combination thereof. Each possibility represents a separate embodiment of the present invention.

According to certain embodiments, the at least one terpene is selected from the group consisting of myrcene, limonene, pinene, linalool, caryophyllene, humulene, and any combination thereof. Each possibility represents a separate embodiment of the present invention.

According to certain embodiments, the at least one flavonoid is of a class selected from the group consisting of flavones, flavonols, isoflavones, flavanones, chalcones, and anthocyanins. Each possibility represents a separate embodiment of the present invention.

Any formulation is as known in the art suitable for efficient delivery of the callus-derived pluripotent cells of the invention to its target activity in a mammalian skin can be used according to the teachings of the present invention.

The at least one dermatologically acceptable carrier can be a cosmetic acceptable carrier or a pharmaceutical acceptable carrier.

According to certain embodiments, the callus-derived pluripotent cells comprised within the skin-care preparation are in a form of a dry powder.

According to certain embodiments, the dry powder is a freeze-dried powder.

According to certain embodiments, the preparation is formulated in a form of a plurality of nanoparticles.

According to some embodiments, the nanoparticle is selected from a group consisting of a liposome, a micelle, a solid-lipid nanoparticle, a cyclodextrin nanoparticle, a dendrimer, a polymeric nanoparticle, a micro/nano-emulsion, and any combinations thereof.

Each possibility represents a separate embodiment of the present invention.

According to certain embodiments, the plant-based skin care preparation of the invention is for use in maintaining and/or promoting skin appearance.

According to certain embodiments, maintaining and/or promoting skin appearance comprises at least one of maintaining and/or promoting skin structure, skin strength, skin cohesion and any combination thereof.

According to certain embodiments, maintaining and/or promoting skin appearance comprises inhibiting skin aging, including skin photoaging.

According to certain embodiments, the plant-based skin care preparation of the invention is for use in the treatment of at least one skin disorders.

According certain embodiments, the skin disorder is selected from the group consisting of acne; alopecia areata; basal cell carcinoma; Bowen's disease; congenital erythropoietic porphyria; contact dermatitis; Darier's disease; dystrophic epidermolysis bullosa; eczema (atopic eczema); epidermolysis bullosa simplex; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; squamous cell carcinoma; Sweet's syndrome; vitiligo; and any combination thereof. Each possibility represents a separate embodiment of the present invention.

According to some embodiments, the skin disorder is an inflammatory skin disorder.

According to certain embodiments, the inflammatory skin disorder is selected from the group consisting of atopic dermatitis, pruritus, and psoriasis. Each possibility represents a separate embodiment of the present invention.

According to certain embodiments, the plant-based skin preparation is produced by a method comprising the steps of:
a. providing at least one callus formed from a sterile Cannabis explant;
b. culturing the plant cells under 2-dimensional (2D) culture conditions to form a callus mass;
c, transferring the callus mass to a liquid medium forming a 3-dimensional (3D) cell suspension culture;
propagating the cells within the medium to form a biomass of callus-derived pluripotent cells; and
e. separating the callus-derived pluripotent cells from the medium.

According to certain embodiments, the Cannabis explant is obtained from a Cannabis plant inflorescence. According to certain embodiment, the explant is obtained from the inflorescence bract. According to certain embodiments, the explant is obtained from bract tissue containing trichomes. According to certain embodiments, the explant is obtained from leaves of sprouting clones of Cannabis.

2D media for forming a callus mass and 3D media for propagating callus-derived pluripotent cells are known in the art. The media (solid in 2D and liquid in 3D) include carbon source and water, typically further include at least one growth regulator hormone, at least one vitamin, at least one mineral and a combination thereof.

According to some embodiments, propagating the cells is further performed under conditions inducing and/or enhancing the production of at least one secondary metabolite. According to certain embodiments, the at least one secondary metabolite is an endogenous metabolite of the explant cells as described hereinabove.

According to certain preferred embodiments, the secondary metabolite is a cannabinoid. The cannabinoids are as described hereinabove.

According to certain embodiments, the method further comprises steps of:
f. encapsulating all or part of the callus-derived pluripotent cells, by means of nanoparticles; and
g. incorporating the nanoparticles in the preparation comprising at least one dermatologically acceptable carrier.

According to yet additional certain aspects, the present invention provides a method for treating at least one skin disorder, comprising topically administering to the skin of a mammalian subject in need thereof an effective amount of the skin-care preparation of the invention.

According to yet further certain aspects, the present invention provides a cosmetic method for maintaining and/or promoting skin appearance, comprising topically administering to the skin of a healthy mammalian subject an effective amount of the skin-care preparation of the invention.

The skin-care preparation, the at least one skin disorder and the skin appearance are as described hereinabove.

These and further aspects and features of the present invention will become apparent from the detailed description, examples and claims which follow.

### BRIEF DESCRIPTION OF THE FIGURES

FIG. 1 is a schematic presentation of a bioreactor production system; and

FIG. 2 is a schematic illustration of the process from plant tissue to large scale production of cell suspension.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

The present invention provides skin care preparations which protects mammalian skin against intrinsic and extrinsic stress factors, maintains and/or promotes health skin status and appearance, and treat skin disorders, particularly inflammatory skin conditions. The cannabis-derived stem cells comprised within the preparations of the invention are effective in protecting mammalian skin stem cells from apoptosis and/or in promoting mammalian skin stem cells proliferation. Without wishing to be bound to any specific theory or mechanism of actions, these activities of the Cannabis-derived stem cells of the invention are attributed to the secondary metabolites present in the cells, particularly cannabinoids, providing the preparation of the invention with the capability of reducing dermal inflammatory conditions, and to primary metabolites with the said cells, particularly WUSCHEL (WUS) transcription factors delaying or preventing skin aging, and/or maintaining and/or promoting skin overall structure, including maintaining or promoting the skin strength and cohesion.

### Definitions

The singular forms “a”, “an”, and “the” include plural referents unless the context clearly dictates otherwise.

Unless the context clearly requires otherwise, throughout the specification, the words “comprise”, “comprising” and the like are to be construed in an inclusive sense as opposed to an exclusive or exhaustive sense; that is to say, in the sense of “including, but not limited to”.

As used herein, the term “about” is to be understood as within a range of normal tolerance in the art, for example within 2 standard deviations of the mean. About can be understood as within ±10%, 9%, 8%, 7%, 6%, 5%, 4%, 3%, 2%, 1%, 0.5%, 0.1%, 0.05%, or 0.01% of the stated value. All values provided herein are understood to be modified by the term about.

The term “Cannabis” is used herein in its broad sense and refers to a plant belonging to this genus, of the family Cannabaceae. According to certain preferred embodiments, the Cannabis plant is selected from the group consisting of Cannabis sativa L. and Cannabis indica. Each possibility represents a separate embodiment of the present invention.

As used herein, the terms “callus” or its plural “calli” refer to a mass of disorganized, undifferentiated plant cells, that grow over a plant tissue samples (explants), a Cannabis explant according to certain embodiments of the present invention. Callus can be produced from a single meristem cell or from a single somatic differentiated cell, the later resuming pluripotency, typically designated as “dedifferentiated cell”. Callus thus comprises a plurality of pluripotent (totipotent) cells, being able to regenerate the whole plant body. Under certain liquid culture conditions, callus cells can be maintained in their pluripotent status.

In living plants, callus cells are those cells that cover a plant wound. In biological research and biotechnology callus formation is induced from explants after surface sterilization and plating onto tissue culture medium in vitro. The culture medium is supplemented with plant growth regulators, such as auxin, cytokinin, and gibberellin, to initiate callus formation or somatic embryogenesis. Callus initiation has been described for all major groups of land plants.

The terms “pluripotent stem-cells” and “pluripotent-cells” are used herein interchangeably and refer to slowly dividing cells giving rise to daughter cells that can either differentiate to new tissues and organs, or remain stem-cells.

As used herein, the term “skin” refers to an outer covering of an animal, in particular a mammal. Mammalian skin is composed of two primary layers, namely, the epidermis, which provides waterproofing and serves as a barrier to infection; and the dermis, which serves as a location for the appendages of the skin.

The epidermis is the outermost layer of the skin. It forms the protective wrap over the body's surface and is made up of stratified squamous epithelium with an underlying basal lamina. Cell types that make up the epidermis include Merkel cells, keratinocytes, melanocytes and Langerhans cells. Stem cell location in the epidermis is divided into basal layer stem cells and interfollicular stem cells.

The dermis is the layer of skin beneath the epidermis that comprises connective tissue. The dermis also comprises many mechanoreceptors (nerve endings) that provide the sense of touch and heat. It comprises the hair follicles, sweat glands, sebaceous glands, apocrine glands, lymphatic vessels and blood vessels.

In certain aspects, the skin care preparations of the invention comprise callus-derived pluripotent stem cells of the genus Cannabis cultured in vitro, wherein the cells have been induced by one or more elicitors including one or more biotic, abiotic and environmental stressors to produce secondary metabolites, wherein such secondary metabolites include cannabinoids, terpenes and flavonoids, all or part of the range of secondary metabolites, found in the naturally grown plant.

In certain preferred embodiments, the callus-derived pluripotent stem cells of the invention comprise a plurality of cannabinoids, wherein the types and the profile of the cannabinoid resemble those of the Cannabis explant from which the callus is formed.

In order to obtain a preparation effective in transforming to the target skin all or most of the essence of the metabolites of the callus-derived pluripotent cells, the cells are in whole or part encapsulated by means of nanoparticles, preferably liposomes.

The pluripotent plant stem cells contain active ingredients suitable for skin care, in healthy as well as in diseased conditions

Further, the invention relates to a method for the in vitro cultivation of pluripotent plant stem cells, as well as to the preparation of such plant cell cultures which are suitable for such applications.

According to certain aspects, the present invention provides a plant-based skin care preparation for a mammalian subject, comprising callus-derived cells of a plant of the genus Cannabis, and at least one dermatologically acceptable carrier, wherein the callus-derived cells are pluripotent cells.

According to certain embodiments, the Cannabis is selected from the group consisting of Cannabis sativa L and Cannabis indica. Each possibility represents a separate embodiment of the present invention.

According to certain embodiments, the callus derived pluripotent cells are capable of maintaining and/or promoting at least one of the growth, vitalization, regeneration, survival, and health of mammalian skin stem cells.

According to certain embodiments, the skin stem cells are epidermal stem cells.

According to certain embodiments, the skin stem cells are dermal stem cells.

As used herein, the expression “healthy stem cells” refers to epidermal stem cells that perform the biological and biochemical activities and functions of wild type epidermal stem cells.

The skin of mammals is a multilaminar system which is continuously revolving. The external layer which is constantly in contact with the outside world is the epidermis. The major task of this specialized tissue is to protect the body against dehydration, lesions and infections. It is composed of four different laminas, which are all formed by a single cell type, keratinocytes. Whereas this cell type is not much differentiated, it nevertheless has its origin in specialized skin stem cells, which are located in the lowermost lamina of the epidermis, the basal lamina.

The term “stem cells (SC)” is used herein with reference to mammalian cells, and refers to uniform undifferentiated cells having the property of constant regeneration and the unique ability of turning into any other cell type by cleavage and differentiation. By said potential, SC are a renewable source of human tissue. Thus, SC has become an important object of medical research for various applications, such as gene therapy, organ transplantation, diabetes, and plastic surgery.

In several papers, successful isolation of such skin stem cells is reported. It has even been demonstrated that skin stem cells may be found in lower tissues of the skin, i.e., in the so-called hair follicle bulge. Contrary to the SC in the basal lamina, these SC are multipotent, i.e., they are capable of differentiating into every tissue type of the skin. The unique properties of plant pluripotent cells have been a recent area of interest in cosmetics, focusing both in developing new cosmetics and studying how these extracts/phytohormones will influence animal skin (see: Roh et al., 2006. Pediatric Research 59 (4):Pt 2, 100-103R; Morasso et al., 2005. Biol. Cell., 97:173-183; Alonso et al., 2003. PNAS, 100, Suppl. 1:11830-11835; Trehan S et al., 2017. Future Sci. OA 3(4), FS0226; Morui M et al., 2014. Acta Poloniae Pharmaceutica 71(5):701-707).

Skin stem cells are crucial in wound healing and the regeneration of skin and hair. However, the capacity of these abilities may be disturbed by genetic problems, environmental influences, and the aging process. Thus, protection of these SC is extremely important.

In exemplary embodiments of the present invention, a callus-derived pluripotent cells obtained from Cannabis plant explant able to protect and stimulate these SC in skin care preparations is developed.

As used herein, the term “inflammatory skin disorders” refers to skin disorders associated with an inflammatory imbalance. Inflammatory skin diseases are one of the most common dermatological problems. Inflammatory skin diseases may be caused by one or more of the following: microbial infection-induced dermatitis; solar dermatitis; atopic dermatitis; and allergic contact dermatitis.

Such inflammatory skin diseases range from simple rashes that occur in combination with itching and redness, to chronic conditions such as dermatitis, eczema, rosacea, seborrheic dermatitis, and psoriasis.

Skin inflammation can be characterized as acute or chronic. Acute inflammation can result from exposure to UV radiation, ionizing radiation, allergens, or from a contact with chemical irritants. This type of inflammation is typically resolved within 1 to 2 weeks with little accompanying tissue destruction. In contrast, chronic inflammation results from a sustained immune cell mediated inflammatory response within the skin itself. This inflammation is long lasting and can cause significant and serious tissue destruction

The process of skin inflammation is complex and is still not completely understood. This is the process during which the body repairs tissue damage and defends itself against harmful stimuli.

Inflammation is provoked by pathogens, noxious mechanical and chemical agents, and autoimmune responses, and it is a complex process during which the body repairs tissue damage and defends itself against harmful stimuli. Inflammation is characterized by such symptoms as redness, swelling, itching, heat, and pain (Ikeda, Y et al., 2008. Molecular nutrition & food research, 52(1):26-42). Under the influence of an inflammatory factor, some intracellular biochemical substances are released from cells. Monocytes and macrophages produce cytokines (regulatory glycoproteins of the immunological system). The basic role of cytokines in inflammatory processes is, inter alia, to activate cells involved in the inflammation (neutrophils, macrophages, and mast cells), enable communication between them, induce the prostaglandin synthesis and affect the synthesis of the C-reactive proteins. Among cytokines one can distinguish pro-inflammatory and anti-inflammatory ones. Predominance of the first type leads to the systemic inflammatory reaction whereas predominance of anti-inflammatory cytokines results in the anti-inflammatory response.

The proinflammatory cytokines cause immune cells to leave the blood and migrate into the skin where they then produce more inflammatory hormones, as well as enzymes, free radicals, and chemicals that damage the skin. The end result of the initial triggering event is the amplification of a large inflammatory response that, while designed to help the skin fight infection from invading bacteria for instance, actually causes considerable damage to the skin.

The skin, in particular the skin keratinocytes, are a potent source of many cytokines. Certain inflammatory skin diseases are associated with overproduction of cytokines, an alteration in cytokine receptors or dysregulation of cytokines. (Sauder, D N., 1990. Journal of Investigative Dermatology 95(5):27S-28S).

The cytokine IL-1 is known to stimulate production of the cytokines IL-6 and IL-8, once a cytokine cascade is set in motion caused by trauma, bacterial toxins or UV light. Such overproduction of cytokines then causes release of other cytokines and an inflammatory response.

Inflammatory skin diseases such as psoriasis and atopic dermatitis can be caused by the overproduction of cytokines. For example, the cytokines IL-1, IL-6 and IL-8 have all been found to be over-expressed in psoriatic plaques, (Sauder, 1990, ibid). In atopic dermatitis, also known as atopic eczema, during the chronic phase, there is activation of TNF-alpha and IL-8 and IL-12 cytokines, (Nedoszytko B. et al., 2014. Advances in Dermatology and Allergology/Postepy Dermatologii i Alergologii, 31(2):84).

These disorders comprise, but are not limited to: acne; alopecia areata; basal cell carcinoma; Bowen's disease; congenital erythropoietic porphyria; contact dermatitis; Darier's disease; dystrophic epidermolysis bullosa; eczema (atopic eczema); epidermolysis bullosa simplex; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; squamous cell carcinoma; Sweet's syndrome; and vitiligo.

In exemplary embodiments of the present invention, a plant formulation of callus-derived pluripotent cells of the genus Cannabis, including Cannabis sativa L. and Cannabis indica, which protects mammalian skin stem cells against intrinsic and extrinsic stress factors, and able to treat these inflammatory skin conditions, as well to maintain and/or promote the overall skin health and appearance, is developed.

The basic principle of cultivation of plant pluripotent cells utilizes the biological fact, as is known to one skilled in the art, that every plant cell has the ability to build up the whole plant from which the pluripotent stem cells originate. This ability is called totipotency and is comparable with the pluripotency of animal SC wherein pluripotent stem cells can divide into most, or all, cell types in an organism, but cannot develop into an entire organism on their own. Therefore, it may be accepted that pluripotent plant cells do have a positive influence on protection and activation of human skin stem cells.

Callus-derived pluripotent plant cells consist of a complex matrix of constituents of salts, acids, polyphenols, sugars, fats, proteins, and other components. In addition to known components there is an unknown fraction of components which possibly is very valuable for cosmetic applications. It is known that raw plant extracts often show a better effect than identified and isolated individual components. Therefore, it is reasonable to use the entire cell or cell lysate for application.

Advantageously, the callus-derived pluripotent cells of the invention, not hitherto known to be used in skin-care preparations, comprise at least one secondary metabolite selected from cannabinoids, flavonoids, and terpenes, preferably cannabinoids. Without wishing to be bound by any specific theory or mechanism of action, the combination of the cells of the skin-care preparation of the present invention being pluripotent stem cells and the presence of secondary metabolites within these cells, preferably cannabinoids, provides for a synergistic beneficial effect of the preparation on mammalian skin, particularly on the stem cells within said skin.

In order to obtain the callus-derived pluripotent cells of the invention including the active ingredients produced therein, special techniques are required since part of them are water-soluble whereas another part is fat-soluble. It has been proposed to process plant cell culture preparations by means of, but not limited to, lyophilization (e.g., International (PCT) Applications publication No. WO 2005/072697; U.S. Application Publication No. 2005/0265953). Thereafter, the lyophilized cells can be pulverized and used in topical preparations.

According to certain embodiments, a method of preparing callus-derived pluripotent cells suitable for use in the preparations of the invention comprises the following main steps:
a. establishment of a stable callus-derived pluripotent cell line;
b. mass cultivation of the pluripotent cells in a suitable bioreactor;
c. recovery of whole cells by dewatering means including, but not restricted to centrifugation, rotary drums, and decantation; and optionally,
d. drying of whole cell mass by means including, but not limited to, freeze drying, spray drying, and heat pump assisted drying.

Optionally, in step (d) the procedure is followed by:
(i) decomposition of the plant cells by high pressure homogenization; and
(ii) stabilization of the substances of content by means including liposomes.
whereby both steps are preferably but not necessarily simultaneously performed as one single step.

According to certain embodiments, the plant-based skin care preparation comprises the callus-derived pluripotent cell at a concentration of from about 0.001% to about 25% w/w based on a dry weight of the total weight of the preparation.

According to certain embodiments, the plant-based skin care preparation comprises the callus-derived pluripotent cell at a concentration of from about 0.01% to about 25% w/w, or from about 0.1% to about 25%, or from about 1% to about 25%, or from about 10% to about 25%, or from about 0.01% to about 20% w/w, or from about 0.1% to about 15%, or from about 1% to about 10%, w/w based on a dry weight of the total weight of the preparation.

According to certain embodiments, the present invention provides an entourage effect of the secondary metabolites that include but not limited to, cannabinoids, terpenes, and flavonoids from the callus-derived pluripotent cells of the invention.

The term “Entourage Effect” as is used in the art refers to a proposed mechanism by which cannabis compounds act synergistically to modulate the overall psychotropic and anti-inflammatory effects of the plant. As used herein, the term further refers to a mechanism by which cannabis compounds found in cannabis-derived pluripotent stem cells act synergistically on mammalian skin, particularly on mammalian skin stem cells.

As used herein, the term “cannabinoids” as used herein refers to a class of diverse chemical compounds that act on cannabinoid receptors on cells that modulate physiological responses in the brain, peripheral nervous and immune systems. The native endocannabinoid ligands (produced naturally in the body by humans and animals), the phytocannabinoids (found in cannabis and some other plants), and synthetic cannabinoids (manufactured chemically) bind to receptors throughout the body and control downstream signal transduction. One example of a cannabinoid is Cannabidiol (CBD), which is a major secondary metabolite in Cannabis (hemp) and non-psychotropic Cannabis extracts. It may have multiple potential applications such as for the treatment of epilepsy and other motor disorders, inflammation, mood and anxiety disorders, sleep dysfunction and eating disorders. CBD is also considered a promising antineoplastic agent on the basis of its in vitro and in vivo activity against tumor cells.

The endocannabinoid system (ECS) regulates many physiological processes involved in relaxation, eating, sleeping, certain inflammatory responses and even cognitive function. There are two types of cannabinoid receptors found throughout the body (CB1 and CB2), but they are most abundant in the brain and immune system respectively. In fact, the CB1 receptor is the most densely populated G-coupled protein receptor in the human brain. New evidence indicates that a cannabinoid-like ligands act on wide variety of biological targets, such as the transient receptor potential cation channel, nuclear receptors and other orphaned G-coupled protein receptors (i.e., TRPV1, PPAR, GPR18 and GPR55), and represents a fascinating area to develop new therapeutic targets. Cannabinoids comprise, but are not limited to: Tetra-hydro-cannabinoids (d9-THC), Tetra-hydro-cannabinoids (d8-THC), Tetra-hydro-cannabinolic acid (THCA-d9), Tetra-hydro-cannabivarin (THCV/THC-C3), Cannabidiol (CBD), Cannabidivarin (CBDV), Cannabigerol (CBG), cannabicyclol (CBL), cannabivarin (CBV), Cannabigerolic acid (CBGA), Cannabinol (CBN), Cannabidiolic acid (CBNA), Cannabichromene (CBC), Cannabichromenic acid (CBCA), cannabichromevarin (CBCV), cannabigerovarin (CBGV), cannabigerol monomethyl ether (CBGM), cannabielsoin (CBE), cannabicitran (CBT), both in the natural carboxylated (acidic) form or in the decarboxylated form.

According to certain embodiments, the preparation comprises at least about 0.05%, at least about 0.06%, at least about 0.07%, at least about 0.08%, at least about 0.09% or at least about 0.1% total cannabinoids out of the total dry weight of the callus-derived pluripotent cells.

According to certain embodiments, the preparation comprises between about 0.05% to about 3%, about 0.75% to about 3%, about 0.1% to about 3% total cannabinoids out of the total dry weight of the callus-derived pluripotent cells.

As used herein, the term “terpenes” refers to plant-derived molecules, also known as isoprenoids and are the largest and most diverse group of naturally occurring compounds that are mostly found in plants. They are responsible for the fragrance, taste, and pigment of plants

The basic molecular formula of terpenes are multiples of that, (C₅H₈)ₙ where n is the number of linked isoprene units. Terpenes are fragrant oils that give cannabis its aromatic diversity.

For example, the terpene linalool is responsible for the aroma and relaxing properties of lavender.

As used herein, the term “flavonoids” refers to a class of polyphenolic secondary metabolites found in plants, and thus commonly consumed in the diets of humans. Chemically, flavonoids have the general structure of a 15-carbon skeleton, which consists of two phenyl rings and a heterocyclic ring.

Flavonoids are an important class of natural products; particularly, they belong to a class of plant secondary metabolites having a polyphenolic structure, widely found in fruits, vegetables, and certain beverages.

Flavonoids can be subdivided into different subgroups depending on the carbon of the C ring on which the B ring is attached and the degree of unsaturation and oxidation of the C-ring. Flavonoids in which the B-ring is linked in position 3 of the C-ring are called isoflavones. Those in which the B-ring is linked in position 4 are called neoflavonoids, while those in which the B-ring is linked in position 2 can be further subdivided into several subgroups on the basis of the structural features of the C-ring: flavones, flavonols, flavanones, flavanonols, flavanols or catechins, anthocyanins and chalcones.

Flavonoids are a class of molecules that are ubiquitous among plants, and several are consumed as nutritional supplements for their antioxidant properties.

According to particular embodiments, the invention relates to the use of callus-derived pluripotent cells grown in-vitro from the genus Cannabis including Cannabis sativa L. and Cannabis indica, wherein these pluripotent cells contain all or part of the range of cannabinoids and terpenes found in the in-vivo grown plant, the prime cannabinoids being taken from the group consisting of but not confined to: tetrahydrocannabinol (THC) (delta-9-THC), cannabidiol (CBD), cannabidivarin (CBDV), cannabigerol (CBG), cannabigervarin (CBGV) and tetrahydrocannabivarin (THCV), whether in the natural carboxylated (acidic) form or in the decarboxylated form, and the prime terpenes from the group consisting of but not confined to myrcene, limonene, pinene, linalool, caryophyllene, and humulene.

Flavonoids can be subdivided into different subgroups depending on the carbon of the C ring on which the B ring is attached and the degree of unsaturation and oxidation of the C-ring. Flavonoids in which the B-ring is linked in position 3 of the C-ring are called isoflavones. Those in which the B-ring is linked in position 4 are called neoflavonoids, while those in which the B-ring is linked in position 2 can be further subdivided into several subgroups on the basis of the structural features of the C-ring: flavones, flavonols, flavanones, flavanonols, flavanols or catechins, anthocyanins and chalcones.

Flavonoids are a class of molecules that are ubiquitous among plants, and several are consumed as nutritional supplements for their antioxidant properties.

According to particular embodiments, the invention relates to the use of callus-derived pluripotent cells grown in-vitro from the genus Cannabis including Cannabis sativa L. and Cannabis indica, wherein these pluripotent plant cells contain all or part of the range of cannabinoids and flavonoids found in the in-vivo grown plant, the prime cannabinoids being taken from the group consisting of but not confined to: cannabidiol (CBD), cannabidivarin (CBDV), cannabigerol (CBG), cannabigervarin (CBGV) and tetrahydrocannabivarin (THCV), whether in the natural carboxylated (acidic) form or in the decarboxylated form, and the prime flavonoids is of class selected from the group consisting of but not confined to flavones, flavonols, isoflavones, flavanones, chalcones, and anthocyanins.

The physiological effects of THC and CBD are affected by the presence of other cannabis-derived molecules, such as additional cannabinoids, terpenes, and flavonoids. Certain terpenes and flavonoids can interact with cannabinoid receptors, and this is thought to be one reason they contribute to the anti-inflammatory and psychotropic effects of cannabis. Consumption of THC and/or CBD in combination with other cannabis-derived molecules can enhance the desired effects of THC and/or CBD, and the combinatory action of cannabis-derived molecules can be referred to as entourage effects. Entourage effects may enhance the therapeutic potential of cannabinoids such as THC and CBD, with respect to pain, inflammation, depression, anxiety, addition, epilepsy, cancer, and infections (Russo E. 2011. British Journal of Pharmacology. 163(7):1344-1364). Entourage effects may also counteract THC side effects, such as dysphoria, and/or may enhance cannabis-induced euphoria.

Entourage effects also play a large role in the distinct effects of different cannabis strains. Entourage effects can contribute to the sedating, energizing, concentration-enhancing, relaxing, and/or other effects induced by particular cannabis strains.

It was found that a non-psychotropic cannabinoid cannabidiol (CBD), exerted anti-acne effects (Olih, A. et al, 2014. The Journal of clinical investigation, 124(9): 3713-3724).

Neither viability or basal sebaceous lipid synthesis were altered, however CBD normalized pro-acne agents and induced seborrhoea-mimicking lipogenesis.

CA 2910206 describes a composition comprising tetrahydrocannabinol (THC) and CBD in combination with a corticosteroid for the treatment of psoriasis.

WO 2010/013240 describes the anti-inflammatory effect of CBD and suggests it may be of use in the treatment of psoriasis and atopic dermatitis.

Tubaro et al. describe the topical anti-inflammatory action of CBD and CBDV (Tubaro A. et al., 2010. Fitoterapia, 81(7), 816-819).

WO 2013/006729 describes a nano-enhanced patch containing CBD which may be used in the treatment of psoriasis.

Wilkinson et al. describes that cannabinoids are able to inhibit human keratinocyte proliferation via a non CB1/CB2 mechanism and have a potential therapeutic value in the treatment of psoriasis (Wilkinson, J. D., & Williamson, E. M. 2007. Journal of dermatological science, 45(2):87-92).

US 2015/086494 describes an anti-inflammatory cream which comprises THC and CBD in addition to hydrocortisone.

GB 2516335 describes the use of phytocannabinoids in the treatment of skin carcinomas.

From the publication WO 2005/072697 cited hereinabove it is known to use lyophilizates of undifferentiated plant cells for depigmenting the skin. This technique calls for the use of lyophilizates of undifferentiated plant cells, in particular of cells of halophile plants. A use for stimulation and protection of skin stem cells from the Cannabis sativa plant is not envisaged. In a further publication, U.S. Pat. No. 9,155,916 the use of undifferentiated plant cells in cosmetic preparations is disclosed. A use of stem cells as cosmetic preparations derived from the Cannabis plant is not envisaged. In a further publication, WO 2018/0263952 the use of one or more cannabinoids in the treatment of inflammatory skin diseases is disclosed. A use of cannabinoids derived from stem cells of the Cannabis plant is not envisaged.

More particularly in some embodiments, the compositions of the present invention are formulated for topical application or intradermal administration. Each possibility represents a separate embodiment.

The terms topical administration or topical treatment or topical therapy or topical application refer hereinafter to application to body surfaces such as the skin or mucous membranes to treat ailments via a large range of classes including, for example, creams, foams, gels, lotions, and ointments. Many topical medications are epicutaneous, meaning that they are applied directly to the skin.

The term intradermal injection, refers hereinafter to superficial injection of a substance into the dermis, which is located between the epidermis and the hypodermis.

For topical or intradermal administration, the compositions of the present invention may be formulated as an oil, a gel, a stick, a lotion, a cream, a milk, an aerosol, a spray, a foam, a mousse, an ointment, liquid drops, nebulized liquid, a liquid wash, an emulsion, a suspension, liposomes, an adhesive patch, and a powder. Each possibility represents a separate embodiment. Currently preferred are compositions formulated as a gel, an ointment, a cream or an emulsion. Each possibility represents a separate embodiment. Encompassed within the present invention are emulgel compositions which comprise an emulsion (e.g., oil-in-water or water-in-oil) and a gel (e.g., a hydrogel or a hydroalcoholic gel) and possess the advantages of both emulsions and gels, for example as being easily spreadable and easily removable. Alternative forms of the compositions of the present invention may also be used including forms which are designed for reconstitution with a suitable vehicle prior to use. Intradermal delivery of the peptides, peptide derivatives or salts thereof through nanoneedles or microneedles for example using a patch as described in Larraneta et al. (Mater. Sci. Eng. R 104: 1-32, 2016) is also contemplated within the scope of the present invention. Optionally, a combination with other skin protective treatments may be used.

A further, preferred embodiment for topical delivery is liposomes.

As used herein, the term “liposomes” refers to closed bilayer structures spontaneously formed by hydrated phospholipids that are widely used as efficient delivery systems for drugs or antigens, due to their capability to encapsulate bioactive hydrophilic, amphipathic, and lipophilic molecules into inner water phase or within lipid leaflets.

As used herein, the term “nanoparticles” refers to particles between 1 and 250 nanometers (nm) in size having a core surrounded by an interfacial layer (the shell). In some embodiments, the nanoparticle is selected from a liposome, a micelle, a solid-lipid nanoparticle, a cyclodextrin nanoparticle, a dendrimer, a polymeric nanoparticle, a micro/nano-emulsion and any combinations thereof.

In some embodiments, the NP is a vessel or a liposome, comprising a lipid bilayer or a single layer (a multilamellar liposome or a unilamellar liposome).

Liposomes can be used to carry and deliver an agent, e.g., a composition described herein, into a cell. Detailed guidance can be found in, e.g., Yarosh et al. (2001) (Lancet 357: 926; and Bouwstra et al. 2002. Adv. Drug Deliv. Rev. 54 Suppl 1:S41). Since transport of materials through the skin barrier is very limited, the technique of producing liposomes for many cosmetic applications was developed (e.g., KR20050091162, KR920005639B, GB2415375, WO2004067012, EP1498420, US2002160064, AU2388099). Application of this technique allows a better penetration of substances into the lower skin laminas. Also, a further advantage of liposomes is the encapsulation of fat-soluble ingredients in the membrane and thus their dispersion in aqueous phases.

There are various methods of liposome production. Main steps of their production comprise dissolving a phospholipid mixture in a suitable solvent (e.g., glycerol or alcohol), intermixing the dissolved lipids with an aqueous phase, applying energy (e.g., by stirring, shaking, pressure or heat) for forming the liposomes. As said above, the form of energy can by pressure. Formation of liposomes by means of high-pressure homogenization is a known technique. Examples for pharmaceutical or cosmetic preparations may be found e.g., in WO9949716, NZ502840 or EP0782847. Interestingly the same technique can be used for solubilizing cells and obtaining their lysate (e.g., DE19918619). Therefore, it is possible to solubilize plant cells of suspension cultures and at the same time to extract the oil- and water-soluble agents into empty liposomes. Thereby, stability of the agents and their transportation into the skin can be improved.

Some embodiments of this invention are directed to topical or intradermal formulations. The anti-aging formulations may include cannabidiols, cannabidiol isomers, cannabidiol analogs, or combinations thereof and a carrier, excipient, diluent, reagent, or combinations thereof.

And in some embodiments, such formulations may further include one or more antioxidants, anti-wrinkling agents, anti-inflammatory agents, emollients, proactants, conditioning agents, and combinations thereof. Such formulations may interrupt or prevent the production of tyrosinase and melanin and may a provide a reduction in fine lines and wrinkles as well as firming and lifting of the skin to reduce notable sagging.

The term “skin care preparation” comprises one or more cosmetically acceptable excipients that facilitate and application to the skin of a mammalian subject.

The excipients included in the preparation typically comprise emulsifiers and emollients, and optionally also a thickener, a buffering or pH adjusting agent, a humectant, and combinations thereof. Each possibility represents a separate embodiment. Additional excipients that may be included comprise a surfactant, an anti-oxidant, a fragrance, a colorant and combinations thereof. Each possibility represents a separate embodiment. The reconstitution compositions of the present invention typically comprise an antimicrobial preservative.

Suitable emulsifiers include, but are not limited to, polyethylene glycol ethers of stearic acid such as steareth-2, steareth-4, steareth-6, steareth-7, steareth-10, steareth-11, steareth-13, steareth-15, and steareth-20, glyceryl stearate, stearyl alcohol, cetyl alcohol, cetearyl alcohol, behenyl alcohol, diethanolamine, lecithin, polyethylene glycols and combinations thereof. Each possibility represents a separate embodiment. Combinations of emulsifiers are available commercially, for example under the trademarks Dow Corning® 5225C (DC 5225C), Montanov™ 68, Emulium® Delta.

Suitable emollients include fats, oils, fatty alcohols, fatty acids and esters which aid application and adhesion, yield gloss and provide occlusive moisturization. Examples include, but are not limited to, silicone oils (for example, those available under the trademarks Dow Corning® 245 (DC 245) and Dow Corning® 246 (DC 246), glycerin, hydrocarbon oils and waxes, including mineral oil, petrolatum, paraffin, ceresin, ozokerite, microcrystalline wax, polyethylene, and perhydrosqualene; triglyceride fats and oils, including those derived from vegetable, animal and marine source including jojoba oil and shea butter; acetoglyceride esters, such as acetylated monoglycerides; ethoxylated glycerides, such as ethoxylated glyceryl monostearate; fatty acids, fatty alcohols and derivatives thereof, such as isopropyl myristate. Each possibility represents a separate embodiment.

Suitable thickeners include, but are not limited to, various polymers such as polyacrylic acid, polymethacrylic acid, acrylamides copolymer, sodium acrylates copolymer, sodium alginate, calcium alginate, magnesium alginate, alginic acid, hyaluronic acid, polyglucuronic acid (poly-α- and -β-1,4-glucuronic acid), chondroitin sulfate, furcellaran, carboxymethylcellulose, polycarboxylic acids, carbomer, bentonite, chitin, chitosan, carboxymethyl chitin, and cross-linked polyacrylate materials available under the trademark Carbopol®, as well as polymers such as hydroxypropyl methylcellulose (HPMC), hydroxypropyl cellulose (HPC), hydroxymethyl cellulose, hydroxyethyl cellulose, methyl cellulose, calcium carboxymethyl cellulose, polyvinylpyrrolidone (povidone, PVP), polyvinyl alcohol, medium to high molecular weight polyethylene glycols (PEG-3350, PEG-6000, etc.), glucosides and tetrasodium etidronate. Each possibility represents a separate embodiment of the present invention.

Suitable buffering or pH adjusting agents include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide. Each possibility represents a separate embodiment.

Suitable humectants include, but are not limited to, glycols such as triethylene glycol, tripropylene glycol, propylene glycol, polypropylene glycols, butylene glycol, polyethylene glycols, sugar alcohols such as sorbitol, hexylene, urea, and collagen. Each possibility represents a separate embodiment.

Examples of cosmetically acceptable antimicrobial preservatives include formaldehyde releasers (e.g., imidazolidinyl urea), parabens (e.g., methylparaben, propylparaben), isothiazolinones (e.g., kathon), phenoxyethanol (e.g., optiphen) and organic acids (e.g., benzoic acid/sodium benzoate, sorbic acid/potassium sorbate). Each possibility represents a separate embodiment of the present invention.

The cosmetic compositions and products of the present invention are useful for skin rejuvenation, improving skin appearance, skin tone and the like. In particular, the cosmetic compositions and products of the present invention are useful in improving various skin conditions, including for example wrinkles, fine lines, skin imperfections, spots including lentigines or solar lentigines, uneven skin tone or texture, UV radiation-induced damaged skin or photodamaged skin, hyperpigmented skin or melasmas, dry skin, sagging skin, rough skin, and any combination thereof.

Cultivation volumes exceeding 10 liter necessitates the use of special bioreactors instead of culture flasks used in smaller volumes. Many different systems are available on the market. Execution of cultivation may be done in, but is not limited to, agitation reactors, bubble columns, loop reactors or newly developed single-use systems suitable for plant cell cultivation. For all these cultures the influence of shearing stress must be monitored since they can damage the cultures and inhibit productivity. Thus, the most important parameter for selecting a suitable reactor system is typically the manner in which the culture is continuously dispersed within the medium.

Moreover, control of the culture is very important. In comparison to cultures of yeast or bacteria, measurement of the biomass is difficult, and the growth of biomass should be measured by means of indirect parameters, such as e.g., consumption of carbon, dropping of conductivity or the pH value or the increase of optical density. Once such a control is established, the end point or the harvest time, respectively, can be determined.

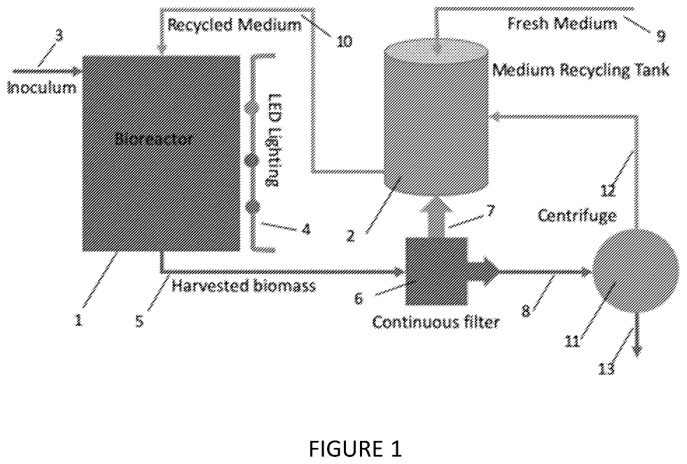

Reference is made to FIG. 1, describing an exemplary bioreactor to be used according to the teachings of the invention. A bioreactor 1 is filled with medium from a medium recycling tank 2 with the composition as described in Example 4 hereinbelow. The medium is inoculated by prepared inoculum 3, the composition of which is described hereinbelow. Secondary metabolites are elicited by the addition of appropriate elicitors. There are many elicitors, both biotic and abiotic, known in the art for inducing the production of secondary metabolites in plant cell suspensions. Yeast extract (YE) showed the most positive results as described in Example 4. The bioreactor 1 is continuously stirred and irradiated with LED lights 4, activated in various configurations, periodicities and wavelengths. At a designated concentration of cells per liter, determined by weight or by cell count, a volume of harvested medium 5 is removed and filtered through a fine mesh filter 6 or other such filtration device such that a filtrate 7 of liquid medium and particulate matter such as bacteria cells pass through while a retentate of plant cells 8 are withheld. The filtrate 7 is passed to the medium recycling tank 2 and sterilized by means of UV lighting or steam. The concentration levels of the growth medium are checked through standard analytical means and adjusted by the introduction of fresh medium 9 or by the addition of specific amounts of any component. Sterilized, reconstituted medium is recycled 10 back into the bioreactor 1. The cellular retentate 8 in the filter unit 6 is washed and further dewatered in a continuous centrifuge device 11. Liquid output 12 from the centrifuge device 11 is recycled to the medium recycling tank 2 and the product biomass 13 is removed for drying or extraction.

The following steps provide an undifferentiated cell line from plant tissue:
a. Choice of a suitable tissue for the induction.
b. Surface sterilization;
c. Cladding of the explants on a suitable solid medium for callus induction;
d. Harvesting the callus grown on the injured surface of the explants;
e. Sub-cultivation of the obtained callus on the same medium until the cells are entirely undifferentiated;
f. Addition of the undifferentiated cells to a suitable liquid medium;
g. Homogenization of the cells in suspension until big cell clump are no longer present; and
h. Sub-cultivation and continuous characterization of the cell suspension.

Fundamental working protocols for plant cell cultures can be found in the standard literature and are well known to those familiar with the art (e.g., Plant Cell Culture: A Practical Approach, Editor P. A. Dixon, 1994, Oxford University Press). Protocols for the work and suitable media for initiating plant cell cultures, e.g., of apples, are described by Nitsch et al., 1970, (Bull. Soc. Bot. Fr., 117:479-492) and Pech et al., 1975 (Bull. Soc. Bot. Fr., 122:183-194). According to these protocols, initiating and maintaining such cultures can be done.

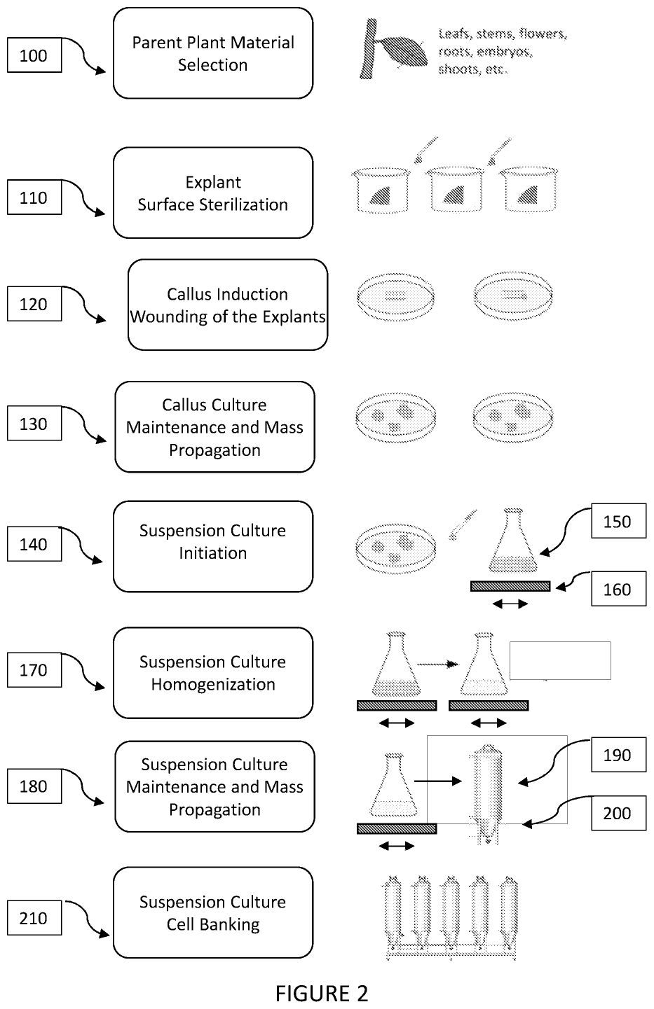

Detailed reference is now made to FIG. 2 which illustrates in broad terms the stages from selecting an initial cutting from an in-vivo plant through to full scale production of undifferentiated stem cells derived from said plant. Selection is made of suitable cuttings from the in-vivo plant (100). As is known by one skilled in the art, the most suitable cutting is from plant tissue that preferentially expresses the secondary metabolites of interest in the elicited stem cells. In the case of the present invention wherein the desired secondary metabolites include one or more cannabinoids, cuttings are taken primarily, but not necessarily, from the bract leaves. Bract leaves are the small leaves that surround the reproductive cells of a female cannabis plant and contain high concentrations of trichomes: glands which secrete cannabinoids such as THC. The cuttings are decontaminated (110) in suitable sterilizing solutions such as sodium hypochlorite. The cuttings are then transferred under aseptic conditions to solid media (120) containing growth promoters as described in more detail below. The cuttings develop into calli (130) containing pluripotent cells and plant cells that have reverted to undifferentiated stem cells. When the calli have matured they are cut and transferred (140) to liquid media in Erlenmeyer flasks (150) continuously stirred by orbital stirrers (160). The viable undifferentiated stem cells in the callus detach from the callus, become a cell suspension and reproduce thus increasing the concentration of cells. The media is filtered (170) to remove clumps of callus material that resist dispersion. As the concentration of cells further increases (180) the medium becomes the inoculum for the bioreactor (190). After transfer into a bioreactor (190), whether disposable, plastic bioreactor, or stainless-steel bioreactor (190) more medium is added. Agitation of the cell suspension in the bioreactors (190) is preferably via air bubbles entering the bioreactor (190) in the area of the lower conus (200). The cell suspension is then transferred to arrays of bioreactors, whether disposable, plastic bioreactors, or stainless-steel bioreactors (210) for large scale propagation of cell matter.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Reference is now made to the following examples, which together with the above descriptions, illustrate the invention in a non-limiting fashion.

### Example 1: Preparation of Callus from Seeds

Germination of sterilized seeds: 1000 seeds of Cannabis sativa were sterilized in batches of approximately 100 seeds. First, the seeds were washed in strainers for 15 min with running tap water. Afterward, the seeds were dipped for 60 seconds in 70% ethanol and then washed with sterilized deionized water for 5 min under a laminar airflow cabinet. Once in the laminar flow hood and following the wash, the seeds were transferred into 500 ml autoclaved Erlenmeyer flasks and sterilized using 12% (v/v) commercial bleach (sodium hypochlorite) for 12 min, followed by three, 5-minute deionized water rinses.

The growth medium was based on the standard Murashige and Skoog's (MS) medium supplemented with sucrose. This medium contains: Mineral components: ammonium nitrate (NH₃NO₃) in an amount of 1650 mg/dm³, potassium nitrate (KNO₃) in an amount of 1,900 mg/dm3, magnesium sulphate (MgSO₄·7H₂O) in the amount of 370 mg/dm³, potassium dihydrogen phosphate in the amount of 170 mg/dm³ (KH₂PO₄), calcium chloride (CaCl₂·2H₂O) in the amount of 440 mg/dm³, potassium iodide (KI) in the amount of 0.83 mg/dm³, boric acid (H₃BO₃) in an amount of 6.2 mg/dm³, manganese (II) sulphate (MnSO₄·4H₂O) in an amount of 22.3 mg/dm³, zinc sulfate (ZnSO₄·7H₂O) in an amount of 8.6 mg/dm³, molybdate (VI) sodium (Na₂MoO₄·2H₂O) in the amount of 0.25 mg/dm³, iron (II) sulphate (FeSO₄·7H₂O) in the amount of 27.8 mg/dm³, disodium edetate (Na₂EDTA-2H₂O) in the amount of 37.3 mg/dm³, copper (II) sulfate (CuSO₄·5H₂O) in the amount of 0.025 mg/dm³, cobalt chloride (II) (CoCl₂·6H₂O) in the amount of 0.025 mg/dm³.

Organic additives: niacin in the amount of 0.5 mg/dm³, pyridoxine HCl in the amount of 0.5 mg/dm³, thiamine HCl in the amount of 0.1 mg/dm³, glycine in the amount of 2 mg/dm³. The ingredients were weighed and dissolved in distilled water. The pH was adjusted to 5.7 with concentrated HCl and NaOH.

Solid ingredients: inositol in the amount of 100 mg/dm³, sucrose in the amount of 30 g/dm³ and agar in the amount of 7 g/dm³ were weighed and added to the previously prepared base. All the ingredients were heated at high temperature until the medium boiled. The solution was autoclaved for 20 min at 120° C. at a pressure of 1 atm. (0.1 Mpa). 30 ml of media were poured into sterile Magenta GA7 vessels. After 24 hours approximately 20 seeds per Magenta were laid out on the solid media in the laminar flow hood. The seeds were illuminated with 40 PAR fluorescent light (μE·m-2·s-1) for 16 hours a day and kept at 22-24° C.

Germination commenced after 1-2 weeks. After 2 to 4 weeks, the health of the germinated seeds was assessed. Seed or seedlings that were stunted, infected or dead were removed.

Initiation of shoot subcultures: Sterile seedlings approximately 6 cm in height with at least four leaves were used to initiate the shoot cultures. In a laminar flow hood, the healthiest sterile sprouts were removed from the surface of the solid medium and the leaves removed from the sprout. The leaves were cut into approximately 3 mm by 3 mm sections and placed on fresh solid medium in sterile Magenta GA7 vessels. The medium was identical to the standard MS medium described hereinabove with the addition of indole-3-acetic acid (IAA) in an amount of 0.2 mg/dm³ as growth promotor to initiate new shoot cultures from the cuttings of the sterile seedlings. The IAA was sterilized with the media in the autoclave. The cuttings were illuminated with 40 PAR fluorescent light (μE·m-2·s-1) for 16 hours a day and kept at 22-24° C.

Propagation of shoot subcultures: After approximately 2-3 weeks the leaf fragments began rooting. After approximately 3-4 more weeks the hypocotyl and the cotyledons began to emerge from the cell mass. After approximately 1-2 further weeks with 4 leaves above the cotyledon a new selection was made for the healthiest clones. At this stage a monogenetic population of sterile clones had been created. They became the starting material for the further stages. This population was propagated periodically every 8-10 weeks.

Initiation of callus cultures: Fragments of shoots (leaf blades) were taken from this subculture of sterile shoot cultures in the manner described hereinabove, collected and placed on nutrient-agar media in sterile Petri dishes. The medium was identical to the standard MS medium described hereinabove with the addition of growth promotors: indole-3-acetic acid (IAA) in an amount of 0.2 mg/dm³ added to the medium before sterilization, and kinetin (KIN) in an amount of 2 mg/dm³ and jasmonic acid (JA) in an amount of 0.2 mg/dm³ both filtered with a 0.22-micron syringe filter and added to the media after cooling down to room temperature. The callus cultures were illuminated with 40 PAR fluorescent light (μE·m-2·s-1) continuously and kept at 22-24° C.

Propagation of callus cultures: Callus tissue was visible around the wounded leaf blades after 3-4 weeks. The callus tissue was friable and green. After a further 2-4 weeks the callus material was cut into 0.5 cm by 0.5 cm pieces and propagated under identical conditions on agar-solidified media in sterilized Petri dishes.

### Example 2: Preparation of Callus from Cuttings

Tissue cuttings (explants) were taken of bract leaves from a mature, flowering cannabis Hemp plant type Futura 75 (CBD 3.145% and THC 0.039%). Bract leaves are the small leaves that surround the reproductive cells of a female cannabis plant and contain high concentrations of trichomes: glands which secrete cannabinoids.

Decontamination and sterilization were started with washing with detergent, followed by washing with 70% ethanol for 3 min, and a rinsing period with sterilized distilled water for 10 min. Thereafter the explants were transferred to a solution of 2% sodium hypochlorite in which the explants were kept for 20 min, after which they were again thoroughly rinsed three times with sterilized distilled water and placed on solid medium in sterilized Petri dishes.

The growth medium was based on the standard Murashige and Skoog's (MS) medium supplemented with sucrose and growth promoters. This medium contains:

Mineral components: ammonium nitrate (NH₃NO₃) in an amount of 1650 mg/dm³, potassium nitrate (KNO₃) in an amount of 1,900 mg/dm3, magnesium sulphate (MgSO₄·7H₂O) in the amount of 370 mg/dm³, potassium dihydrogen phosphate in the amount of 170 mg/dm³ (KH₂PO₄), calcium chloride (CaCl₂·2H₂O) in the amount of 440 mg/dm³, potassium iodide (KI) in the amount of 0.83 mg/dm³, boric acid (H₃BO₃) in an amount of 6.2 mg/dm³, manganese (II) sulphate (MnSO₄·4H₂O) in an amount of 22.3 mg/dm³, zinc sulfate (ZnSO₄·7H₂O) in an amount of 8.6 mg/dm³, molybdate (VI) sodium (Na₂MoO₄·2H₂O) in the amount of 0.25 mg/dm³, iron (II) sulphate (FeSO₄·7H₂O) in the amount of 27.8 mg/dm³, disodium edetate (Na₂EDTA-2H₂O) in the amount of 37.3 mg/dm³, copper (II) sulfate (CuSO₄·5H₂O) in the amount of 0.025 mg/dm³, cobalt chloride (II) (CoCl₂·6H₂O) in the amount of 0.025 mg/dm³.

Organic additives: niacin in the amount of 0.5 mg/dm³, pyridoxine HCl in the amount of 0.5 mg/dm³, thiamine HCl in the amount of 0.1 mg/dm³, glycine in the amount of 2 mg/dm³. The ingredients were weighed and dissolved in distilled water. The pH was adjusted to 5.7 with concentrated HCl and NaOH.

Solid ingredients: inositol in the amount of 100 mg/dm³, sucrose in the amount of 30 g/dm³ and agar in the amount of 7 g/dm³ were weighed and added to the previously prepared base.

Growth Promotors: indole-3-acetic acid (IAA) in an amount of 0.2 mg/dm³ added to the medium before sterilization, and kinetin (KIN) in an amount of 2 mg/dm³ and jasmonic acid (JA) in an amount of 0.2 mg/dm³ both filtered with a 0.22 micron syringe filter and added to the media after cooling down to room temperature.

All the ingredients were heated at high temperature until the medium boiled. The solution was autoclaved for 20 min at 120° C. at a pressure of 1 atm. (0.1 Mpa). 30 ml of media were poured into sterile Petri dishes. After 24 hours the decomtaminated and sterilized explants were laid out on the solid media in the laminar flow hood. The seeds were illuminated with 40 PAR fluorescent light (μE·m-2·s-1) for continuously and kept at 22-24° C.

Propagation of callus cultures: Callus tissue was visible around the wounded leaf blades after 3-4 weeks. The callus tissue was friable and green. After a further 2-4 weeks the callus material was cut into 0.5 cm by 0.5 cm pieces and propagated under identical conditions on agar-solidified media in sterilized Petri dishes.

### Example 3: Elicitation of Callus Cultures with Yeast Extract

Yeast extract (YE) is known in the art as an efficient elicitor in both plants grown in-vivo and cell tissue culture grown in-vitro. YE (Sigma-Aldrich) was dissolved in purified water (1:1), filtered through a 0.22-micron syringe filter and added dropwise to the solid medium wherein the callus culture was growing in a manner described above hereinabove. The dripping rate was such that the free liquid was fully absorbed into the agar matrix, this volume being approximately 2-5 ml per addition per Petri dish. Treatment took place once a day. The treatment commenced after the callus had grown beyond the area of the leaf cuttings for a period of 3 weeks. After this period of treatment, the calli were collected.

One gram of calli biomass, obtained as described hereinabove, was homogenized for 10 min with a benchtop homogenizer (Bullet Blender Tissue Homogenizer) and extracted in one step with 4 mL of chloroform and 4 mL of methanol/water (1/1) by vortexing for 30 sec and sonication for 1 min. The mixture was then centrifuged at 3,000 rpm, 25° C. for 20 min. The two solvent layers were collected separately. Extraction was repeated and the resulting extracts were combined with the first extraction. The combined extracts were evaporated until dry. The chloroform extract was subjected to HPLC analysis. The dry residue was redissolved in 1 ml Methanol and filtered with 0.22 μm PES Syringe Filter. Samples were stored at −18° C. and warmed up to 4° C. before analyses.

### HPLC

Cannabinoid composition was determined using High Performance Liquid Chromatography (HPLC). Analyses were performed on an Agilent Technologies (Waldbronn, Germany) modular model 1100 system, A Poroshell 120 C18 column (Poroshell 120 SB-C18, 2.1×100 mm, 2.7 m, (Agilent, Milano, Italy). The column was used with a mobile phase composed of 20% formic buffer and 80% acetonitrile (ACN). The flow rate was 1.2 mL/min. The run time was 13.2 min. The column temperature was set at 30° C. The sample injection volume was 20 μL. Cannabinoids were identified by comparing both the retention time and the absorption spectrum at 220 nm with those of authentic purified standards. Three injections were performed for each sample.

### Standards

Standard curves were derived from six independent injections of six concentrations (2, 10, 50, 200, 1000 and 5000 ng/ml) of THCA, THC, CBGA, CBG, CBDA and CBD. Linearity in peak area versus injected amount for all six compounds was found in the concentration range between 10 and 1000 ng/ml with high reproducibility and accuracy.

### Results

There are many elicitors, both biotic and abiotic, known in the art for inducing the production of secondary metabolites in plant cell suspensions. Those that showed positive results for cannabinoid production include: methyl jasmonate, light regulation, hexanoic acid, GABA, ethephon, tyrosine, phenylalanine, salicylic acid, yeast extract. Of these, YE, showed the largest effect. YE at concentrations of 1:1 in purified water dripped on to solid media supporting cannabis calli yielded total concentrations of cannabinoids 0.14% of which 0.13% was identified as CDB/CBDA and 0.002% as THC/THCA compared to CBD/CBDA of 3.145% and THC/THCA of 0.039% in the mother plant and non-detectable quantities of cannabinoids in calli grown according to Example 1 and 2 hereinabove without elicitation.

### Example 4: Elicitation of Cell Suspension Cultures

The fragmented callus tissue from Example 2 was allowed to grow and was then transferred to sterilized 250 ml Erlenmeyer flasks with 100 ml of liquid medium (MS) including the growth promoters identical to those described in Example 2. The flasks were placed on orbital shakers. After 2-3 weeks incubation the cell suspension was filtered to remove callus clumps and an additional volume of 50 ml of medium was added. After a further 3 weeks the concentration of the cell suspension was sufficiently high and the 150 ml cell suspension was transferred to sterile 500 ml Erlenmeyer flasks and the medium volume increased to 300 ml. YE (Sigma-Aldrich) was dissolved in purified water (1:1), filtered through a 0.22 micron syringe filter and 10 ml added to the cell suspension medium periodically. The commencement and period of YE addition was varied from one time addition after removal of clumps, to daily addition of 10 ml after removal of clumps. After a further growth period of 3 weeks the cell suspension was filtered, washed, and dried in a freeze drier. 1 gm of dried Cannabis stem cells was pulverized in a ball bearing homogenizer (Bullet Blender Tissue Homogenizer) and extraction performed as described in Example 3 hereinabove. HPLC was performed as described in Example 3 hereinabove. Results showed that YE elicitation of 10 ml after clump separation yielded the highest increase in cannabinoids: Total CBD—0.95%, and total THC 0.08%.

### Example 5: Induction of Cell Line, Biomass Production and Processing

Scale-up to large bioreactor: Starting at an inoculum concentration of about 1 g/l the culture is allowed to increase cell concentration to about 10-12 g (Dry Weight (DW)/l at which point it is transferred to a 100-liter bioreactor. After 2-3 weeks when the cell concentration has again reached 10-12 DW g/l the inoculum is transferred to a full-size bioreactor of 1250 liters.

When the culture reaches 10-12 g DW/l (about 1 week), it is harvested continuously at about 20% of the volume per 24 hours. This method of harvesting, as opposed to batch harvesting, allows the average cell concentration to remain constant and thereby optimizes productivity.

The growth of the suspended cell culture is primarily heterotrophic in that it utilizes sugar, namely sucrose, as a carbon, source rather than CO₂ and photosynthesis. It is known in the art that the chlorophyll receptors even though they are not necessary for photosynthesis are still functional as photo-triggers for growth of primary and secondary metabolites. To this end, the suspended cell culture is grown in light accessible single-use, sterilized plastic bioreactors. Alternatively, the suspended cell culture is grown in light accessible multiple-use stainless steel, sterilizable bioreactors.

### Example 6: Preparation of a Liposomal Extract

In order to obtain a formulation containing the whole essence of the cultivated cells, the cells are solubilized by means such as liposomes. The main component of this method is the use of high-pressure homogenization of the whole cell broth together with a liposome preparation. The great advantage of this method is its simple and low-cost application.

In detail, the method comprises the following steps:
a. Addition of a suitable liposome preparation to the cell broth;
b. Addition of a suitable preservative agent;
c. Addition of suitable antioxidants;
d. Mixing the substances; and
e. High pressure homogenization.

All preservative agents of natural or synthetic origin allowed for cosmetics, such as for example, phenoxyethanol, benzoic acid, propionic acid, alcohol or silver chloride, can be used as preservative agents.

In order to additionally protect the extract from oxidation, antioxidants, such as for example, ascorbic acid or tocopherol, may be added.

The described method allows the addition of still further substances useful in the preparation of cosmetic products. Once all compounds are added, the mixture has to be stirred in order to dissolve the preservative agents and other components. This may be done e.g., by means of a paddle mixer, a homogenization rod or by pumping through static mixing elements.

The subsequent high-pressure homogenization pursues to objectives:
a. Destruction of the cell membranes in order to release extractable substances; and
b. Generation of finely dispersed liposomes contained the fat- and water-soluble fractions of the cells.

Suitable high-pressure homogenizers are commercially available on the market. The principle of the reaction chamber has to be selected from different possibilities and has to be previously tested. The number of passages through the reaction chamber necessary for a disintegration of all cell membranes or reaching a desired homogeneity of the extract has to be tested as well.

Afterwards, the extract obtained in this manner can directly be incorporated into cosmetic preparations, such as for example, creams, soaps, lotions, gels or hair serums. If the extract is to be used as semi-finished goods a supplemental thickening is possible. All thickening agents of natural or synthetic origin allowed for cosmetics can be used as thickening agents.

The cannabinoids of such stem cells include any of a broad class of compounds that are known to interact with cannabinoid receptors and encompass endocannabinoids (produced naturally in the body by animals), the phytocannabinoids (found in cannabis and some other plants), and synthetic cannabinoids (manufactured artificially). Example cannabinoids include, but are not limited to, tetrahydropyran analogs, such as, 19-tetrahydrocannabinol, 18-tetrahydrocannabinol, 6,6,9-trimythel-3-pentyl-6H-dibenzo[b,d]pyran-1-ol, 3-(1,1-dimethylheptyl)-6,6a7,8,10,10a hexahydro-1-lhydroxy-6,6-dimythel-9H-dibezo[b,d]pyran-9-ol, (−)-(3S,4S)-7-hydroxy-delta-6 tetrahydrocannabinol-1,1-dimethylheptyl, (+)-(3S,4S)-7-hydroxy-A-6-tetrahydrocannabinol, and A8-tetrahydrocannabinol-11-oic acid, piperidine analogs, such as, [(6S,6R,9R,10aR) 5,6,6,7,8,9,10,10a-octahydro-6-methyl-1-3-[(R)-1-methyl-4-phenylbutoxy)-1,9 phenanthridinediol 1]-acetate), aminoalkylindole analogs, such as, (R)-(+)-[2,3-dihydro-5-methyl-3 (4-morpholinylm-ethyl)-pyrrolo[1,2,3,-de]-1,4-benzoxazin-6-yl)]-1-naphthelenyl-methanone, open pyran-ring analogs, such as, 2-[3-methyl-6-(1-methylethenyl-2-cyclohexen-1-yl]-5-pentyl-1,3 benzendi-ol, and 4-(1,1-dimethylheptyl)-2,3′-dihydroxy-6′-a-(3-hydroxypropyl)-1′,-2′,3′,4′,5′,6′ hexahydrobiphenyl, lipophilic alkylamides, such as, dodeca-2E,4E,8Z,10E/Z-tetraenoic-acid isobutylamide, cannabinoid mimetics, salts, solvates, metabolites, and metabolic precursors of these compounds and combinations thereof. In some embodiments, the cannabinoids may be derived plants including hemp.

After cultivation, the whole cell broth was mixed with a dispersion containing empty liposomes of a size of about 50 nm. The mixture was then four times high pressure homogenized at a pressure of about 1200 bar (1.2*108 N m-2) resulting in a finely dispersed extract.

### Example 7: Exemplary Formulations

Exemplary formulations of the present invention are outlined in examples a-e below:

a. Vanishing Cream

The percentage refers to the total quantity (weight/weight)

| | | |
|---|---|---|
| Oily phase 1: | Alkyl benzoates | 10% |
| | Dimeticone | 3% |
| | Archidyl glycosides | 3% |
| | Myristyl glycoside | 2% |
| Oily phase 2: | Polyacrylamides | 1% |
| Aqueous phase: | Demineralized water | 71% |
| | Glycerol | 5% |
| | Phenoxyethanol | 1% |

Oily phase 1 and the aqueous phase were heated at 80° C. and blended. The mixture was chilled to 60° C. Then oily phase 2 was added, and the mixture was blended. The mixture was chilled to 30° C. 4 percent of the extract described in Example 4 was added and the mixture was blended again.

b. Liquid Balm for the Scalp

The percentage refers to the total quantity (weight/weight).

| | |
|---|---|
| Ethanol | 0.5% |
| Urea | 5.0% |
| Propylene glycol | 0.5% |
| Carbomer | 0.4% |
| Bisabololene | 0.1% |
| PEG-60 | 0.6% |
| D-Panthenol 75% | 0.5% |
| Sodium hydroxide 30% | 0.4% |
| Plant cell extract of Example 4 | 1% |
| Water | filling up to 100% |

c. Intensive Face Mask

The percentage refers to the total quantity (weight/weight).

| Phase | Ingredient | Amount |
|---|---|---|
| Aqueous phase 1 (W1) | Water | filling up |
| | | to 100% |
| | Citric acid | 0.6% |
| | Sodium benzoate | 0.5% |
| Aqueous phase 2 (W2) | D-Panthenol 75% | 0.7% |
| Oily phase 1 (O1) | Cetearyl alcohol | 4.5% |
| | Dicocoylethyl hydroxyethlmonium | 3% |
| | methosulfate | |
| | Distearoylethyl hydroxetylmonium | 1.5% |
| | methosulfate | |
| | Dicapryryl ether | 1% |
| | Gycerol stearate | 1% |
| Oily phase 2 (O2) | Amino dimethicone | 0.3% |
| Plant extract (A) | Plant cell extract of Example 4 | 2% |

### Instruction for Preparation:

Aqueous phase 1 is mixed and heated to 75° C. Shortly before mixing with oily phase 2 aqueous phase 2 (panthenol) is added. Oily phase 1 is heated to 75° C., and shortly before mixing oily phase 2 (aminodimethicone) is added. The combined aqueous and oily phases are mixed and homogenized. The mixture is chilled to 30° C., and phase A (plant extract) is added.

d. Eye Cream

The percentage refers to the total quantity (weight/weight).

| Phase | Ingredient | Amount |
|---|---|---|
| Aqueous phase 1 (W1) | Water | filling up |
| | | to 100% |
| | Citric acid | 0.6% |
| | Glycerol | 5% |
| | Butylene glycol | 5% |
| | Galacto arabinane | 0.3% |
| | Parabens in phenoxyethanol | 0.8% |
| Oily phase 1 (O1) | Polyglyceryl-3-methylglucose | 2.5% |
| | distearate | |
| | Hydrogenated polyisobutene | 3% |
| | Vegetable oil | 4% |
| | Dicapryryl ether | 3% |
| | Behenyl alcohol | 2% |
| | Dimethicone | 0.5% |
| Oily phase 2 (O2) | Maize phosphates | 1% |
| | Dimethicone | 0.5% |
| Plant extract (A) | Plant cell extract of Example 4 | 2% |

### Instruction for Preparation:

Aqueous phase is mixed and heated to 80° C. Oily phase 1 is heated to 80° C., and oily phase 2 is added. The combined aqueous and oily phases are mixed and homogenized. The mixture is chilled to 30° C., and phase A (plant extract) is added, and the blend is mixed again.

### Example 8: Effect of Stem Cells of Cannabis sativa L. on Stressed Skin

The following dermatological test is carried out for products prepared in accordance with the present invention.

Introduction: In normal skin the tumor suppressor gene p53 is upregulated by several types of stress, e.g., DNA damage (induced by UV radiation, IR radiation, or chemical agents, such as hydrogen peroxide), oxidative stress, or osmotic shock. The protein p53 plays an important role in the cell cycle as transcription regulator. In old skin this gene is no more upregulated but rather down regulated by stress (Benjamin C L et al. 2007. Toxicol Appl Pharmacol. 224(3):241-8).

### Test Product:

2.0% of stem cells of Cannabis sativa L.
Cells: Normal human dermal fibroblasts (NHDF) used at 10th passage

Procedure: Fibroblasts are stressed for 2 hours with culture medium containing 600 mole of H₂O₂. For recovery, the cells are incubated for 72 hours with a medium containing, or not containing (control), 2% of stem cells of Cannabis sativa L. cultivated according to Example 4 hereinabove. After the incubation time, mRNA is extracted and transcribed into 33P-labeled cDNA via reverse-transcription. These labeled cDNA targets are hybridized to an “old skin” specific minichip. This minichip contains about 150 genes specific for skin aging. The content of labeled genes on the minichip is measured.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-37. (canceled)

38. A plant-based skin care preparation for a mammalian subject comprising callus-derived undifferentiated cells of a plant of the genus Cannabis, and at least one dermatologically acceptable carrier, wherein the callus-derived cells are pluripotent cells.

39. The plant-based skin care preparation of claim 38, wherein the Cannabis is selected from the group consisting of Cannabis sativa L. and Cannabis indica.

40. The plant-based skin care preparation of claim 38, wherein the callus-derived undifferentiated pluripotent cells are capable of maintaining and/or promoting at least one of growth, vitalization, regeneration, survival, and health of mammalian skin cells.

41. The plant-based skin care preparation of claim 38, wherein the concentration of the callus-derived undifferentiated pluripotent cells is from about 0.001% to about 25% w/w based on a dry weight of the total weight of the preparation.

42. The plant-based skin care preparation of claim 38, wherein the callus-derived undifferentiated pluripotent cells are cultured in a medium, forming a cell suspension.

43. The plant-based skin care preparation of claim 42, wherein the cell suspension is maintained under conditions inducing or enhancing the production of at least one secondary metabolite within the callus-derived pluripotent cells.

44. The plant-based skin care preparation of claim 42, wherein said preparation comprises callus-derived pluripotent cells separated from the medium.

45. The plant-based skin care preparation of claim 38, wherein the callus-derived pluripotent cells comprise at least one secondary metabolite, wherein the at least one secondary metabolite is endogenous to the Cannabis plant from which said cells are obtained.

46. The plant-based skin care preparation of claim 45, wherein the at least one secondary metabolite is selected from the group consisting of cannabinoids, terpenes, flavonoids, and any combination thereof.

47. The plant-based skin care preparation of claim 46, wherein said preparation comprises at least 0.05% total cannabinoids out of the total dry weight of the callus-derived pluripotent cells.

48. The plant-based skin care preparation of claim 46, wherein the at least one cannabinoid is selected from the group consisting of cannabidiol (CBD), cannabinol CBN, cannabigerol (CBG), cannabidiolic acid (CBDA), cannabichromene (CBC), cannabicyclol (CBL), cannabivarin (CBV), cannabidivarin (CBDV), cannabigervarin (CBGV), cannabichromevarin (CBCV), cannabigerovarin (CBGV), cannabigerol monomethyl ether (CBGM), cannabielsoin (CBE), cannabicitran (CBT), tetrahydrocannabinol (THC) tetrahydrocannabivarin (THCV), and any combination thereof.

49. The plant-based skin care preparation of claim 38, wherein the callus-derived pluripotent cells comprise WUSCHEL (WUS) transcription factors.

50. The plant-based skin care preparation of claim 38, wherein the callus-derived undifferentiated pluripotent cells comprised within the skin-care preparation are in a form selected from the group consisting of a dry powder and a wet paste.

51. The plant-based skin care preparation of claim 38, wherein said preparation is formulated in a form of a nanoparticle.

52. The plant-based skin care preparation of claim 51, wherein the nanoparticle is selected from a group consisting of a liposome, a micelle, a solid-lipid nanoparticle, a cyclodextrin nanoparticle, a dendrimer, a polymeric nanoparticle, a micro/nano-emulsion, and any combinations thereof.

53. The plant-based skin care preparation of claim 38, wherein said preparation is produced by a method comprising the steps of:
a. providing at least one callus formed from a sterile Cannabis explant;
b. culturing the callus under 2-dimensional (2D) culture conditions to form a callus mass;
c. transferring the callus mass to a liquid medium forming a 3-dimensional (3D) cell suspension culture;
d. propagating the cells within the medium to form a biomass of callus-derived pluripotent cells; and optionally,
e. separating the callus-derived pluripotent cells from the medium,
thereby formulating the callus-derived pluripotent cells into a plant-based skin care preparation.

54. The plant-based skin preparation of claim 53, wherein the method further comprises the steps of encapsulating all or part of the callus-derived undifferentiated pluripotent cells, by means of nanoparticles; and incorporating the nanoparticles in the preparation comprising at least one dermatologically acceptable carrier.

55. A method for treating at least one skin disorder, comprising topically administering to the skin of a mammalian subject in need thereof an effective amount of the plant-based skin-care preparation of claim 38, wherein said skin disorder is an inflammatory skin disorder selected from the group consisting of acne; alopecia areata; basal cell carcinoma; Bowen's disease; congenital erythropoietic porphyria; contact dermatitis; Darier's disease; dystrophic epidermolysis bullosa; eczema (atopic eczema); epidermolysis bullosa simplex; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; squamous cell carcinoma; Sweet's syndrome; vitiligo; and any combination thereof.

56. A cosmetic method for maintaining and/or promoting skin appearance, comprising topically administering to the skin of a healthy mammalian subject an effective amount of the skin-care preparation of claim 38, wherein said maintaining and/or promoting the skin appearance includes at least one of maintaining and/or promoting skin structure, skin strength, skin cohesion and any combination thereof.

57. The cosmetic method of claim 56, wherein maintaining and/or promoting the skin appearance comprises at least one of maintaining and/or promoting skin structure; maintaining and/or promoting skin strength; maintaining and/or promoting skin cohesion; inhibiting skin aging and any combination thereof.
